(19) United States
(12) Patent Application Publication
(10) Pub. No.: US 2020/0039831 A1
(43) Pub. Date: Feb. 6, 2020

(54) MULTIPLE-PRESSURE PROCESS FOR THE PRODUCTION OF AMMONIA

(71) Applicants: THYSSENKRUPP INDUSTRIAL SOLUTIONS AG; thyssenkrupp AG

(72) Inventors: Evgeni GORVAL, Dortmund (DE); Reinhard HEUN, Herdecke (DE); Joachim JOHANNING, Oberhausen (DE); Klaus NÖLKER, Dortmund (DE)

(73) Assignees: THYSSENKRUPP INDUSTRIAL SOLUTIONS AG, Essen (DE); thyssenkrupp AG, Essen (DE)

(21) Appl. No.: 15/735,636

(22) PCT Filed: Jun. 9, 2016
(86) PCT No.: PCT/EP2016/063089

§ 371 Date: Dec. 12, 2017

(30) Foreign Application Priority Data
Jun. 12, 2015 (DE) 10 2015 210 801.5

(51) Int. Cl. C01C 1/04; C01B 3/02; C07C 1/12; C07C 1/04

(52) U.S. Cl. CPC C01C 1/047; C01B 3/025; C07C 1/0485; C07C 1/12; C01C 1/0488

(57) ABSTRACT

A process and a device for the production of ammonia at different pressure levels may involve removing gases that are inert (inert gases) or harmful with regard to ammonia synthesis from the process in a process step before the ammonia synthesis so that enrichment of these is decreased or suppressed. For example, with respect to a gas mixture that includes hydrogen, nitrogen, water, methane, carbon monoxide, and carbon dioxide, at least part of the water, at least part of the methane, at least part of the carbon monoxide, and at least part of the carbon dioxide may be removed from the gas mixture before the synthesis of the ammonia occurs.

The present invention relates to a process and a device for the production of ammonia at different pressure levels, wherein gases which are inert with regard to the ammonia synthesis (inert gases), are preferably removed from the process at a comparatively early stage, so that enrichment thereof is reduced or even completely suppressed.

In the production of ammonia, the synthesis gas as well as hydrogen and nitrogen usually additionally contains inert gases such as methane and noble gases, which impair the yield of ammonia. In these processes, fresh synthesis gas is usually firstly compressed to high pressure in multiple stages. Then the compressed, fresh synthesis gas is fed into a loop which is passed through one or more catalyst-filled reactors in which ammonia is generated. In the loop, a separation system is provided, through which the ammonia generated is removed from the loop in liquid form.

The inert gases are only soluble in low concentration in ammonia, and are therefore only to a small extent withdrawn together with the ammonia. In order to avoid enrichment of the inert gases in the loop, in these conventional processes a part of the gas circulated in the loop is constantly removed as purge gas. From this removed purge gas, residues of ammonia are then scrubbed out, and hydrogen and optionally also nitrogen are separated and recovered, for example via membranes or by fractionation at low temperatures. The remaining inert gases, in particular methane, argon and helium are discarded or utilized in other ways, in particular for heating purposes. The recovered hydrogen and optionally also the recovered nitrogen are mixed with the fresh synthesis gas before the compression and in this manner utilized.

DE 100 57 863 A1 and DD 225 029 A3 disclose processes for the production of ammonia from fresh synthesis gas, which apart from hydrogen and nitrogen contains inert constituents, in at least two reaction systems, wherein the synthesis of ammonia from synthesis gas is effected consecutively in different synthesis systems. During this, inert gas constituents are separated via a purge gas stream and discharged.

However, it is energetically unfavorable to discharge purge gas from the loop, since thereby large quantities of gas are subjected to a pressure loss during the separation and must thereafter be expensively recompressed. For this reason, an enrichment of inert gases from originally 1 to 2 vol. % in the fresh synthesis gas up to 10 to 20 vol. % within the recirculated gas is often accepted, although this is inevitably attended with the disadvantage that the partial pressures of hydrogen and nitrogen lie considerably lower than in a synthesis loop which contains little or no inert gases. For this reason, the catalysts volumes and the reactors containing them must be sized markedly larger than would be necessary without inert gases in the synthesis loop.

In ammonia synthesis, a gas mixture, which as well as the unreacted hydrogen and nitrogen contains the ammonia formed and the inert gases, is formed in the reactor from the synthesis gas. At the outlet from the reactor, the ammonia generated is in gaseous form. In order to separate the ammonia from the product gas, it is condensed so that it can be withdrawn liquid from the loop. Since the dew point of ammonia depends on its partial pressure, the condensation of ammonia is favored by a high synthesis pressure, a high ammonia concentration and a low temperature. A high ammonia concentration can be achieved with large catalyst volumes and low concentrations of inert gas. A high synthesis pressure means corresponding energy expenditure for the synthesis gas compression and a low cooling temperature requires the appropriate cooling devices for the circulated gas.

In order to be able to condense the generated ammonia within the loop, a synthesis pressure in the range from 150 and 280 bar is usually selected. As well as the improved reaction conditions, this relatively high pressure offers the advantage that a major part of the ammonia already condenses at relatively high temperatures, such as can sometimes be achieved even with water cooling. Since the circulated gas which is recycled to the reactor should have as low an ammonia concentration as possible, an additional deep freeze loop is usually connected downstream of the water cooling in order to condense out further ammonia with still lower temperatures.

The lower the synthesis pressure lies, the more the proportion of the heat decreases which can be removed by water or air cooling and that proportion of heat which has to be removed by deep freezing increases correspondingly. While the compression cost for the synthesis loop decreases with decreasing synthesis pressure, the compression cost for the cooling loop increases, since more cold is needed for the separation of the ammonia generated in the synthesis loop. In the ammonia synthesis at lower synthesis pressure, the proportion of condensation before the deep freezing is increased by the fact that a very low content of inert gas is set via a high purge gas flow. Here also, a lower content of inert gas increases the ammonia concentration and thus the dew point.

DE 10 055 818 A1 discloses a process for the catalytic production of ammonia from a nitrogen-hydrogen gas mixture comprising the formation of synthesis gas from natural gas and oxygen-rich gas in an autothermal reactor, the catalytic conversion of carbon monoxide into hydrogen, the removal of carbon monoxide, carbon dioxide and methane and the passing of the nitrogen-hydrogen gas mixture to a catalytic synthesis of ammonia.

The invention is based on the problem of providing an improved process for the production of ammonia.

A first aspect of the invention relates to a process for the production of ammonia comprising the steps

(a) preparation of a gas mixture comprising hydrogen, nitrogen, water, methane, carbon monoxide, carbon dioxide, in some cases argon, and optionally helium,
(b) removal of at least a part of the water, at least a part of the methane, at least a part of the argon, at least a part of the carbon monoxide and at least a part of the carbon dioxide from the gas mixture, preferably by cooling and/or scrubbing and/or adsorption, and optionally setting of the ratio of hydrogen to nitrogen required for the ammonia synthesis to about 3,
(c) optionally compression of the gas mixture to elevated pressure, preferably to a pressure in the range from 60 to 130 bar, more preferably from 90 to 115 bar, synthesis of ammonia from at least a part of the hydrogen and from at least a part of the nitrogen which is contained in the gas mixture, and removal of at least a part of the synthesized ammonia from the gas mixture by cooling,
(d) compression of the gas mixture to a pressure which is higher than the pressure in step (c), preferably to a pressure in the range from 150 to 280 bar, (d′) optionally either removal of at least a part of the synthesized ammonia from the gas mixture, preferably by cooling, or optionally synthesis of ammonia from at least a part of the hydrogen and from at least a part of the nitrogen which is contained in the gas mixture, and removal of at least a part of the synthesized ammonia from the gas mixture, preferably by cooling,
(e) combination of the gas mixture with a recirculated gas mixture comprising hydrogen, nitrogen and ammonia and optionally the other components not completely removed in step (b); here the combination according to step (e) can be effected preferably after or more preferably before the compressor stage in the loop which the gas mixture traverses in the process according to the invention,
(f) synthesis of ammonia from at least a part of the hydrogen and from at least a part of the nitrogen which is contained in the gas mixture, removal of at least a part of the synthesized ammonia from the gas mixture by cooling, compression of the gas mixture to an elevated pressure, and return of the gas mixture as recirculated gas mixture to step (e).

In step (a) of the process according to the invention, a gas mixture is prepared which contains hydrogen, nitrogen, water (steam), methane, and argon, and usually also still other constituents, such as for example carbon monoxide, carbon dioxide and optionally traces of helium. The gas mixture is preferably obtained as synthesis gas from hydrocarbons, preferably from natural gas, water in the form of steam and air or oxygen by reforming and subsequent gas purification. Suitable processes for generating such a synthesis gas are known to those skilled in the art and concerning this reference can for example be made in its entirety to A. Nielsen, I. Dybkjaer, Ammonia—Catalysis and Manufacture, Springer Berlin 1995, Chapter 6, pages 202-326. The main constituent by volume of this gas mixture is preferably hydrogen, wherein the nitrogen content can optionally also be relatively high, depending on whether air, oxygen-enriched air or even pure oxygen was used in its production.

The gas mixture prepared in step (a) of the process according to the invention can as synthesis gas already have been subjected to conventional processing measures such as for example helium removal, natural gas desulfurization, conversion of carbon monoxide to carbon dioxide and carbon dioxide scrubbing. However, even after implementation of these processing operations, the gas mixture contains hydrogen, nitrogen, water (steam), methane and argon, and usually also still other constituents such as residual quantities of carbon monoxide, residual quantities of carbon dioxide and sometimes traces of helium.

In a preferred embodiment, the gas mixture prepared in step (a) is produced from hydrocarbons, preferably from natural gas, water in the form of steam and air or oxygen by reforming, wherein the hydrocarbons preferably contain no argon or only a small quantity of argon. It is known to those skilled in the art that natural gas in various regions often also contains various quantities of argon and sometimes no argon whatever. If the reforming is effected using pure oxygen, e.g. from an air fractionation plant, or with highly oxygen-enriched air, then these preferably also contain no argon or only a small quantity of argon, so that also no significant quantities of argon are introduced by this route. In this embodiment, the gas mixture prepared in step (a) therefore at most contains only small quantities of argon and in step (b) the removal of argon from the gas mixture is sometimes not necessary—in step (b) then essentially the removal of the other gases, in particular of at least a part of the methane, of the carbon monoxide and of the carbon dioxide, takes place.

In another preferred embodiment, the gas mixture prepared in step (a) is produced from hydrocarbons, preferably from natural gas, water in the form of steam and air or oxygen by reforming, wherein the hydrocarbons preferably already contain a significant quantity of argon. If in addition the reforming is performed using air or air only oxygen-enriched to a slight extent, then additional argon is introduced thereby. In this embodiment, the gas mixture prepared in step (a) therefore contains significant quantities of argon and in step (b) then the removal of at least a part of the methane, at least a part of the argon and of the carbon monoxide and of the carbon dioxide is effected.

Preferably the gas mixture prepared in step (a) contains inter alia carbon monoxide. If the removal in step (b) is effected using cryogenic methods, such as for example by nitrogen scrubbing or strong cooling (see U.S. Pat. No. 3,442,613) then between steps (a) and (b) residues of carbon monoxide, and in the process also optionally carbon dioxide, are preferably largely converted to methane by methanation. For the removal of carbon monoxide by strong cooling and condensation, especially low temperatures are necessary so that carbon dioxide in some cases present already condenses beforehand in the cooling. If on the other hand the removal in step (b) is effected by pressure swing adsorption (PSA), then the methanation can preferably be omitted. Suitable processes for hydrogenation of carbon monoxide to methane are known to those skilled in the art. Through the methanation, the content of carbon monoxide, and in the process also in some cases carbon dioxide, in the gas mixture is decreased and the content of methane in the gas mixture increased. This has on the one hand the advantage that carbon monoxide, like also carbon dioxide, as a catalyst poison must in any case only be contained in very small quantities in the gas mixture during the ammonia synthesis. On the other hand, however, at atmospheric pressure methane has a boiling point higher by ca. 30° C., so that in the preferred step (b) methane can be separated from the gas mixture with less expense than carbon monoxide.

In step (b) of the process according to the invention, the removal of at least a part of the water, at least a part of the methane, at least a part of the argon, at least a part of the carbon monoxide and at least a part of the carbon dioxide from the gas mixture is preferably effected by cooling and/or scrubbing. In this, the gas mixture is preferably cooled so strongly that water, methane and argon condense out under the given conditions and thus can be separated from the gas phase by phase separation.

In a preferred embodiment, the carbon monoxide at least partially separated from the gas mixture in step (b) is passed into a CO converter, in which the carbon monoxide is oxidized to carbon dioxide and water reduced to hydrogen. Suitable CO converters are known to those skilled in the art. In another preferred embodiment, the removed carbon monoxide is not passed directly into a CO converter, but instead is mixed with the educt from which the gas mixture prepared in step (a) is produced. If the educt is natural gas, so that the gas mixture prepared in step (a) is synthesis gas produced from natural gas, then the removed carbon monoxide is preferably mixed with natural gas and the mixture thus obtained then compressed, before the synthesis gas is produced therefrom by reforming.

In a preferred embodiment, the removal is effected by cooling by means of cryogenic methods, particularly preferably by nitrogen scrubbing. This embodiment is particularly preferred when the gas mixture prepared in step (a) contains argon. The gas mixture is preferably cooled to a temperature at which water, methane, argon and optionally carbon monoxide are no longer gaseous under the given conditions, but hydrogen still is. The gas mixture is preferably cooled to temperatures of less than −150° C., more preferably less than −170° C. and in particular ca. −190° C. Under these conditions, carbon monoxide and methane at least partially condense out; water and carbon dioxide are already solid at markedly higher temperatures. In a preferred embodiment, the removal is effected by nitrogen scrubbing at a gas mixture pressure in the range from 30 to 100 bar, preferably ca. 90 bar. This has the advantage that such low temperatures do not then have to be reached in order to effect the condensation of the gases to be removed. In addition, this has the advantage that the gas mixture at this pressure can optionally also be passed directly to the ammonia synthesis in step (c) without additional compression, i.e. a further compressor which is connected upstream of the ammonia reactor can also optionally be omitted. The nitrogen scrubbing has the advantage that the removal of the gases can be effected practically completely or almost completely.

If the removal is effected by a nitrogen scrubber, the gas mixture is preferably firstly conventionally cooled, for example in a heat exchanger. Optionally, the gas mixture is firstly strongly cooled (see U.S. Pat. No. 3,442,613), in order then subsequently to be subjected to nitrogen scrubbing.

In another preferred embodiment, the removal is effected only by strong cooling (see U.S. Pat. No. 3,442,613), i.e. no scrubbing as in the nitrogen scrubbing takes place, but instead a strong supercooling, whereby methane is condensed out practically completely and in some cases argon at least partially by the condensation. This embodiment is particularly preferred when the gas mixture prepared in step (a) contains no argon or only a small quantity thereof. In the case of an autothermal reformer or secondary reformer operated with oxygen, the gas mixture (synthesis gas) contains no argon or only a small quantity thereof, insofar as no argon or only a small quantity thereof is contained in the natural gas which is used for generation of the gas mixture (synthesis gas), so that by strong cooling practically all inerts, i.e. methane, in some cases argon, optionally carbon monoxide and optionally carbon dioxide can already be removed.

If the removal is effected by strong cooling (e.g. to −185° C.) (see U.S. Pat. No. 3,442,613), the gas mixture is preferably firstly conventionally cooled, for example in a heat exchanger.

In a further preferred embodiment, the removal is effected by pressure swing adsorption (PSA). Pressure swing adsorption is known to those skilled in the art. In this, molecular sieves which essentially only pass hydrogen and sometimes traces of argon, often ca. 50 ppm, are preferably used. This embodiment is also particularly preferred when the gas mixture prepared in step (a) contains no argon or only a small quantity thereof.

If the removal is effected by pressure swing adsorption, a methanation of carbon monoxide and/or carbon dioxide present in the gas mixture is preferably omitted.

The gas mixture obtained by the preferred step (b) preferably has a

content of water of at most 0.05 mol. %, more preferably at most 0.02 mol. % and in particular at most 0.01 mol. %, and/or
content of carbon dioxide of at most 5 ppm by volume, more preferably at most 2 ppm by volume and in particular at most 1 ppm by volume, and/or
content of carbon monoxide of at most 5 ppm by volume, more preferably at most 2 ppm by volume and in particular at most 1 ppm by volume, and/or
content of methane of at most 0.05 mol. %, more preferably at most 0.02 mol. % and in particular at most 0.01 mol. %, and/or
content of argon of at most 0.05 mol. %, more preferably at most 0.02 mol. % and in particular at most 0.01 mol. %.

In a further preferred embodiment, the gas mixture prepared in step (a) of the process according to the invention consists only of the pure hydrogen and pure nitrogen, i.e. neither water (steam), nor methane, nor argon, nor carbon monoxide, nor carbon dioxide nor helium, or the content of methane, carbon monoxide and carbon dioxide is so low that the discharge of purge gas in step (f) of the process according to the invention is not necessary. Preferably in this case the pure hydrogen and the pure nitrogen are prepared from external sources. In this embodiment, step (b) is skipped, so that step (c) is performed after step (a), preferably directly following step (a). Since no inert gases are present in the gas mixture, these also cannot enrich. Hence no enrichment has to be accepted, nor is any removal necessary.

Depending on the ratio of hydrogen and nitrogen in the gas mixture obtained by step (b), step (b) optionally additionally includes the adjustment of the ratio of hydrogen to nitrogen required for the ammonia synthesis to about 3.

In step (c) of the process according to the invention, the gas mixture is preferably compressed to elevated pressure, preferably to a pressure in the range from 60 to 130 bar, more preferably 90 to 115 bar. In a preferred embodiment, a compression to a pressure in the range from 150 to 180 bar is even already performed. This pressure is then the synthesis pressure at which the gas mixture is passed into an ammonia reactor. However according to the invention, in view of the in any case sometimes already comparatively high pressures of the gas mixture prepared in step (a) (FrontEnd), it is also possible to feed the gas mixture into the ammonia reactor without additional compression. In this ammonia reactor, ammonia is synthesized from at least a part of the hydrogen and from at least a part of the nitrogen which is contained in the gas mixture. Preferably, the ammonia reactor comprises at least one catalyst bed which is traversed by the gas mixture not purely axially, but instead predominantly radially, preferably from outside inwards. Preferably during this the catalyst bed is not cooled, but instead the synthesis is performed adiabatically. After this at least a part of the ammonia is removed from the gas mixture by cooling, for which the gas mixture preferably firstly passes through a heat exchanger and then a condensing device. In the process, the gas mixture is cooled, preferably to temperatures of less than −15° C., more preferably less than −25° C. and in particular ca. −35° C., preferably however not less than −79° C., so that under the given conditions ammonia condenses out and thus can be removed from the gas phase by phase separation.

In step (d) of the process according to the invention, the gas mixture is compressed to a pressure which is higher than the pressure in step (c), preferably to a pressure in the range from 150 to 280 bar. In another preferred embodiment, the pressure in step (d) is higher than 100 bar. In a particularly preferred embodiment, both the pressure in step (c) and also the pressure in step (d) are each higher than 100 bar, with the pressure in step (d) being higher than the pressure in step (c).

In a preferred embodiment, step (d) of the process according to the invention can include as substep (d′) after the compression of the gas mixture, two preferred alternatives:

In a first preferred alternative, substep (d′) comprises the removal of at least a part of the synthesized ammonia from the gas mixture, preferably by cooling.

In a second preferred alternative, substep (d′) comprises the synthesis of ammonia from at least a part of the hydrogen and from at least a part of the nitrogen which is contained in the gas mixture, and the removal of at least a part of the synthesized ammonia from the gas mixture, preferably by cooling. For this, substep (d′) then comprises the introduction of the gas mixture into a further ammonia reactor, in which ammonia is synthesized from at least a part of the hydrogen and from at least a part of the nitrogen which is contained in the gas mixture. Preferably, the further ammonia reactor comprises a catalyst bed which is traversed by the gas mixture not purely axially, but instead predominantly radially, preferably from outside inwards. Preferably during this the catalyst bed is not cooled, but instead the synthesis is performed adiabatically.

In both preferred alternatives, preferably at least a part of the ammonia from the gas mixture is removed by cooling, for which the gas mixture preferably firstly passes through at least one heat exchanger and then a condensing device. During this, the gas mixture is cooled. The gas mixture is preferably cooled to temperatures of less than −15° C., more preferably less than −25° C. and in particular ca. −35° C., preferably however not less than −79° C., so that ammonia under the given conditions condenses out and so can be removed from the gas phase by phase separation.

In step (e) of the process according to the invention, the gas mixture is combined with a recirculated gas mixture comprising hydrogen, nitrogen and ammonia and optionally other components not completely removed in step (b).

In a preferred embodiment, step (e) of the process according to the invention before the combination comprises the removal of at least a part of the ammonia from the gas mixture by cooling. For this, the gas mixture preferably firstly passes through a heat exchanger and then a condensing device. In the process, the gas mixture is cooled. The gas mixture is preferably cooled to temperatures of less than −15° C., more preferably less than −25° C. and in particular ca. −35° C., preferably however not less than −79° C., so that ammonia under the given conditions condenses out and so can be removed from the gas phase by phase separation. Since however this removal does not take place completely, the recirculated gas mixture usually still contains at least traces of ammonia.

In step (f) of the process according to the invention, the gas mixture is passed into an additional ammonia reactor. In this additional ammonia reactor, ammonia is synthesized from at least a part of the hydrogen and from at least a part of the nitrogen which is contained in the gas mixture at a higher synthesis pressure than in step (c). Preferably, the additional ammonia reactor comprises at least one catalyst bed which is traversed by the gas mixture not purely axially, but instead predominantly radially, preferably from outside inwards. Preferably during this the catalyst bed is not cooled, but instead the synthesis is performed adiabatically. After this, at least a part of the synthesized ammonia is removed from the gas mixture by cooling, for which the gas mixture preferably firstly passes through at least one heat exchanger and then a condensing device. In the process, the gas mixture is cooled. The gas mixture is preferably cooled to temperatures of less than −15° C., more preferably less than −25° C. and in particular ca. −35° C., preferably however not less than −79° C., so that ammonia under the given conditions condenses out and so can be removed from the gas phase by phase separation. After this, the gas mixture is again compressed to an elevated pressure, preferably to a pressure in the range from 150 to 280 bar, and returned to step (e) as recirculated gas mixture.

Preferably in step (f) of the process according to the invention, the gas mixture is returned to step (e) in a closed loop, during which ammonia and optionally further substances such as for example hydrogen and nitrogen dissolved in the ammonia or residual quantities of inert gas are removed from the closed loop under the conditions of the ammonia removal, however purge gas which contains inert gases is not discharged in a separate step. Accordingly, in this preferred embodiment further gas mixture is fed into the closed loop via step (e), and the ammonia removal is the only step in which constituents from the gas mixture are discharged, in particular ammonia, but optionally also comparatively small quantities of other substances such as hydrogen and/or nitrogen and/or methane and/or argon and/or helium.

It was surprisingly found that through the process according to the invention, in particular through the preferred step (b), the enrichment of inert gas in the recirculated gas mixture can be reduced or suppressed so far that a separate discharge (purge) can be omitted, without at the same time having to accept the disadvantages otherwise associated with the enrichment of the inert gases. Preferably the concentration of carbon monoxide, carbon dioxide and water is decreased at the latest in step (b) so far that the catalyst which is used for the synthesis of ammonia is not impaired. The concentration of CO and CO₂ can for example be adequately reduced by methanation. If on the other hand the removal is for example effected by pressure swing adsorption (PSA), the methanation can be omitted. Thus the optionally performed discharge of the inert gases together with the ammonia in the course of the removal of the ammonia by cooling in step (f) suffices to discharge optionally present residual quantities of inert gas from the system and in this manner permanently to maintain the content of inert gas in the recirculated gas mixture vanishingly low. As a result of this, smaller catalyst volumes can be used with higher yields of ammonia, which makes the process more profitable overall. A markedly smaller design of the equipment, pipelines and fittings also becomes possible.

Further, it was surprisingly found that the process according to the invention can be operated such that after the first compressor through which the gas mixture flows, optionally in step (c) or even beforehand, the gas mixture can already be compressed to a pressure in the range of preferably 150 to 180 bar. In this case, the process optionally manages overall with two compressor stages steps (c) and (d) and a further compressor stage in the loop for step (f) to overcome pressure losses, whereas conventional processes render at least one further compressor stage necessary.

Steps (a) to (f) of the process according to the invention are preferably performed in alphabetical order, but not inevitably directly following one another. Thus it is absolutely possible and even preferred that further measures are effected before, between or after individual steps or within individual steps.

Preferably in step (c) and/or in step (d) of the process according to the invention, the gas mixture before the compression is firstly optionally strongly cooled and only after that compressed. The cooling of the gas mixture is effected, as soon as it is water-free, preferably with heat exchangers to temperatures below 0° C., preferably to −16° C. or lower. It was surprisingly found that in this manner the capacity of the plant with regard to the overall yield of ammonia can be considerably increased, since with a lower entry temperature into the ammonia reactor, a greater quantity of hydrogen and nitrogen enters the ammonia reactor and can be converted to ammonia. Preferably the heat liberated in the cooling of the gas mixture can partly be used to generate steam and/or to preheat boiler feed water.

In a preferred embodiment, the synthesis of ammonia takes place in ammonia synthesis units which comprise one or more catalyst beds, wherein the gas mixture cools down between the catalyst beds. Preferably the heat released by cooling between the catalyst beds is partly used to generate steam and/or to preheat boiler feed water.

In a particularly preferred embodiment, step (c) of the process according to the invention is repeated at least once, optionally even more often, e.g. twice, three times or four times, before the implementation of step (d), wherein in each repetition of step (c) the gas mixture is compressed to a pressure which is higher than the pressure previously in step (c), and wherein thereafter in step (d) the gas mixture is compressed to a pressure which is higher than the pressure in the last repetition of step (c). In order to enable the repetition of step (c), the implementation preferably takes place by means of several consecutively connected ammonia synthesis units, wherein the ammonia reactors of the ammonia synthesis units are preferably all positioned together in one pressure vessel or each individually in several, consecutively connected pressure vessels. Preferably the gas mixture is cooled each time between the passage through the individual ammonia reactors, in order partly to remove ammonia and to achieve a higher conversion to ammonia. Suitable reactors are known to those skilled in the art, for example from EP 1 339 641.

Preferably the gas mixture prepared in step (a) of the process according to the invention has a relative molar ratio of hydrogen to nitrogen of more than 3:1, preferably of more than 5:1, and the preferred step (b) of the process according to the invention after the removal comprises the enrichment of the gas mixture with nitrogen, preferably to a relative molar ratio of hydrogen to nitrogen of ca. 3:1, as is desirable for the ammonia synthesis that follows. Preferably the gas mixture is enriched with nitrogen which is prepared by air fractionation.

Preferably, the gas mixture prepared in step (a) of the process according to the invention is produced by reforming from hydrocarbons, preferably from natural gas, where the reforming is effected in a reformer which is preferably operated with pure oxygen, with oxygen-enriched air or with air. Preferably in one of the steps of the process according to the invention the gas mixture is subjected to a CO conversion and/or optionally subsequently a carbon dioxide scrubbing. If the reforming is effected in conventional primary and secondary reformers, the reformer is preferably operated with air. Suitable measures are known to those skilled in the art. Preferably the reformer is a two-stage steam reformer, a steam methane reformer (SMR) or an autothermal reformer (ATR). In this, the pure oxygen, the oxygen-enriched air or the air can be fed into the second stage of the steam reformer or directly into the autothermal reformer, wherein it is also possible that firstly a mixing with water and/or hydrocarbons, preferably with natural gas takes place. These measures lead overall to relief of the FrontEnd of the ammonia plant, i.e. to relief of the generation of the synthesis gas (gas mixture) which is prepared in step (a) of the process according to the invention. Autothermal reformers are preferred according to the invention, since for design reasons the alternative two-stage steam methane reformers are uneconomic at high plant capacities and high pressures.

Preferably the reforming is effected at elevated pressure, so that the gas mixture generated (synthesis gas) which is prepared in step (a) already has a comparatively high pressure, for example of at least 70 bar, more preferably at least 80 bar and in particular at least 90 bar. The elevated reforming pressure also has inter alia the advantage that during the reforming a large quantity of carbon dioxide is produced, which following the production of ammonia is preferably converted to urea with ammonia. Preferably the reformer is an autothermal reformer which is preferably operated at a pressure of at least 100 bar. In reforming at elevated pressure, there in some cases arises the disadvantage of a high methane content in the gas mixture generated in the reforming. This disadvantage can in some cases be compensated by the fact that after a nitrogen scrubbing the gas mixture can be used directly for the synthesis of ammonia and does not have to be further compressed.

It was surprisingly found that in the process according to the invention a removal of at least a part of the originally contained carbon dioxide can be economically advantageously effected by physical CO₂ scrubbing. A CO₂ scrubber is preferably a component of the gas purification which is traversed after the reforming in the production of synthesis gas from hydrocarbons. Since with increasing pressure and decreasing temperatures the efficiency of physical CO₂ scrubbing improves in comparison to chemical CO₂ scrubbing, the process according to the invention preferably includes an least partial removal of the carbon dioxide contained in the gas mixture by physical CO₂ scrubbing. Suitable measures for chemical and physical CO₂ scrubbing are known to those skilled in the art. Preferably such a CO₂ scrubber is connected downstream of a CO conversion.

In a preferred embodiment, the parts removed in step (b) are
(i) passed to the reformer and there used as fuel gas, or mixed with the hydrocarbons which are reformed in the reformer, or
(ii) split into at least three substreams, wherein a first substream is enriched with carbon monoxide, a second substream with carbon dioxide and a third substream with methane, and wherein
   the first substream passes through a CO conversion,
   the second substream is mixed with the carbon dioxide stream formed in a carbon dioxide scrubber, and
   the third substream is mixed with the hydrocarbons which are reformed in the reformer.

Preferably the steps (i) and (ii) are combined. Preferably the third substream is mixed with the hydrocarbons which are reformed in the reformer and/or passed to the reformer and there used as fuel gas.

If in one of the steps of the process according to the invention the gas mixture is subjected to a carbon dioxide scrubbing, the second substream is preferably mixed with the carbon dioxide stream of this carbon dioxide scrubbing.

A fuel gas is converted in a reformer with formation of heat and waste gas and its use is known to those skilled in the art. Preferably, the heat formed in the conversion is used for heating the reformer.

The splitting of the parts removed in step (b) can be effected via all splitting devices known to those skilled in the art. In this, the removed parts are preferably split such that the first substream has a content of carbon monoxide of at least 90 vol. %, more preferably at least 95 vol. % or at least 99 vol. %. Preferably the second substream has a content of carbon dioxide of at least 90 vol. %, more preferably at least 95 vol. % or at least 99 vol. %. Preferably the third substream has a content of methane of at least 90 vol. %, more preferably at least 95 vol. % or at least 99 vol. %. Preferably the substreams are compressed after their splitting. Preferably the parts removed in step (b) are in liquid form. If the parts removed in step (b) are in liquid form, the compression is preferably effected with pumps. The low temperature of the parts removed in step (b) can be used for the cooling of processes within the process according to the invention or of processes of other processes. For example, the low temperature of the parts removed in step (b) can be used for the cooling of the gas mixture in the course of the removal of the ammonia in step (f).

Preferably oxygen and nitrogen are prepared by air fractionation, wherein at least a part of the oxygen is fed into the reformer in gaseous form and at least a part of the liquid nitrogen is used for the cooling in the preferred step (b).

The ammonia reactor in step (c), the further ammonia reactor in step (d) and/or the additional ammonia reactor in step (d) preferably each comprise at least one catalyst bed through which the gas mixture in each case flows not purely axially, but instead predominantly radially, preferably from outside inwards. The especially preferred combination according to the invention, of
the preparation of the gas mixture in step (a) by reforming, preferably by autothermal reforming of hydrocarbons, preferably from natural gas, water in the form of steam and oxygen or oxygen-enriched air from an air fractionation plant, and
the removal of at least a part of the water, at least a part of the methane and optionally of at least a part of the argon from the gas mixture in step (b) by cooling, preferably with cryogenic methods, in particular by nitrogen scrubbing,
   with operation of the process at different pressure levels is particularly advantageous economically when the plant capacities are comparatively very high, e.g. enable production of at least 4000 tonnes/day of ammonia, so that large gas volumes are converted.

In order to avoid an increase in the pressure losses associated with high plant capacities, and thereby inter alia to reduce the load on the stage of the synthesis gas compressor which compresses the recirculated gas mixture, radial flow catalyst beds are advantageous in comparison to axial flow catalyst beds. Preferably the catalyst beds are also not cooled, but instead the synthesis is preferably operated adiabatically. This enables better process control, in particular with high plant capacities, for example of at least 4000 tonnes/day of ammonia.

Further, it is preferred according to the invention to cool (chill) the gas mixture before each compressor stage. If ammonia is used as the coolant, then a cooling for example to ˜−30° C. takes place. If nitrogen is used as the coolant, then the gas mixture can be cooled to still lower temperatures. This precooling decreases the load on the subsequent compressor stage and makes it possible to increase the throughput volumes of gas, which is in particular also advantageous with regard to high plant capacities for example of at least 4000 tonnes/day ammonia.

As regards plant capacities preferred according to the invention of at least 4000 tonnes/day of ammonia, the second housing of the compressor for the gas mixture for step (d) can sometimes be limiting. In order to decrease the load on this, it is preferred to remove as much ammonia as possible from the gas mixture in step (c). This is in particular possible at elevated pressure which favors the ammonia synthesis, since then the condensation of the ammonia already begins at higher temperatures. In contrast, however, at higher pressure the efficiency of the reforming worsens—the content of non-reformed methane in the gas mixture prepared in step (a) increases. The process according to the invention now has the advantage that the removal of the methane in step (b) can be effected so efficiently, for example by nitrogen scrubbing, that a capacity-related increase in the content of methane presents no problem, but instead can be easily addressed by the removal in step (b).

A further preferred possibility for decreasing the load on the synthesis gas compressor in step (d) is obtained if a drying unit for the removal of water before a, usually second, compressor stage is omitted. Such a drying unit is comparatively cost intensive and is attended by undesired pressure losses. Inert gases and/or catalyst poisons such as water vapor are already removed by step (b) of the process according to the invention. Preferably the load on the synthesis gas compressor in step (d) is additionally decreased through the removal of inerts such as argon and methane from the gas mixture in step (b) and/or by the use of a synthesis gas in step (a) which comprises exclusively pure hydrogen and pure nitrogen.

The process according to the invention, on implementation of the preferred step (b) leads to a marked decrease in the load on the synthesis gas compressor in comparison to conventional processes. Further, the gas volume recirculated in steps (e) and (f) is markedly reduced, since no inert gases become enriched. This at the same time leads to a lesser enrichment of ammonia before the loop reactor and the discharge of purge gas can be completely eliminated. This leads overall to a marked increase in the possible capacity of the plant, without the individual components having to be sized larger for this.

In a preferred embodiment,
the gas mixture prepared in step (a) is produced by reforming of hydrocarbons which contain no argon or only a small quantity of argon, and
the reforming is effected in an autothermal reformer which is operated with pure oxygen or with an oxygen/nitrogen mixture which contains no argon or only a small quantity of argon which are prepared by air fractionation, and
in step (a) the gas mixture is subjected to a CO conversion and a carbon dioxide scrubbing, and
carbon monoxide and or carbon dioxide in some cases remaining in the gas mixture is converted to methane by methanation and the methane removed from the gas mixture in step (b) by cooling, and
after the removal in step (b) the gas mixture is enriched with nitrogen, with the nitrogen being prepared by air fractionation.

A further aspect of the invention relates to a process for the production of urea, comprising the process for the production of ammonia, wherein at least a part of the ammonia produced in the process according to the invention is converted to urea with carbon dioxide. Preferably at least 40 vol. % of the ammonia produced in the process according to the invention is converted to urea with carbon dioxide, more preferably at least 50 vol. %, at least 60 vol. %, at least 70 vol. %, at least 80 vol. %, at least 90 vol. % or at least 99 vol. %. Also preferably the whole of the ammonia produced in the process according to the invention is converted to urea with carbon dioxide.

In a preferred embodiment, the gas mixture prepared in step (a) is produced by reforming of hydrocarbons in an autothermal reformer with formation of carbon dioxide, and the ammonia produced in the process according to the invention is at least in part converted to urea with the carbon dioxide formed in the autothermal reformer. In the reforming of hydrocarbons in an autothermal reformer, the quantity of carbon dioxide which arises during the reforming can be controlled by variation of reaction parameters such as for example the pressure or steam/carbon ratio. Preferably in the autothermal reforming just so much carbon dioxide is produced that the ammonia produced in the process according to the invention preferably can be completely converted to urea with carbon dioxide.

A further aspect of the invention relates to a device for the production of ammonia comprising the operatively connected elements
(a) a device for the preparation of a gas mixtures comprising hydrogen, nitrogen, water, methane, carbon monoxide, carbon dioxide and in some cases argon,
(b) at least a first removal device for removal of CO₂ and/or CO from the gas mixture,
(c) at least one further removal device for removal of at least a part of the water, at least a part of the methane, optionally of at least a part of the carbon monoxide, optionally at least a part of the carbon dioxide, and optionally at least a part of the argon from the gas mixture,
(d) means for enriching the gas mixture with nitrogen,
(e) one or more, consecutively connected ammonia synthesis units which each comprise an ammonia reactor for synthesis of ammonia from at least a part of the hydrogen and from at least a part of the nitrogen which is contained in the gas mixture; one or more heat exchangers for cooling the gas mixture and a condensing device for removal of at least a part of the ammonia,
(f) a compressor which is designed to compress the gas mixture to a pressure which is higher than the pressure in the previously traversed ammonia synthesis unit(s),
(g) a device for combining the gas mixture with a recirculated gas mixture comprising hydrogen, nitrogen and ammonia,
(h) an ammonia synthesis unit comprising an ammonia reactor for the synthesis of ammonia from at least a part of the hydrogen and from at least a part of the nitrogen which is contained in the gas mixture, one or more heat exchangers for cooling the gas mixture, and one or more condensing devices for removal of at least a part of the ammonia, a compressor for compressing the gas mixture to a higher pressure, in order to compensate the pressure loss in the loop; and means for recirculating the gas mixture to the device for combining the gas mixture.

Preferably here the at least one first removal device of the device according to the invention comprises a CO₂ scrubber and/or a methanation unit.

Preferably the at least one further removal device further comprises an N₂ scrubber.

Particularly preferably the device according to the invention for the production of ammonia comprises the following operatively connected elements:

a device for preparation of a gas mixture comprising hydrogen, nitrogen, water, methane, in some cases argon, carbon monoxide and carbon dioxide and optionally helium,
optionally a reformer for generation of the gas mixture from hydrocarbons, preferably natural gas, water and oxygen,
optionally a CO conversion unit for the conversion of CO to CO₂,
optionally a CO₂ scrubber for removal of carbon dioxide from the gas mixture,
optionally a methanation unit for the conversion of carbon monoxide to methane,
a removal device for removal of at least a part of the methane, water and argon, and in addition also at least a part of the CO or CO₂ from the gas mixture, preferably by N₂ scrubbing,
optionally one or more heat exchangers for cooling the gas mixture,
optionally an air fractionation plant, means for the transfer of oxygen or oxygen-enriched air to the reformer and means for the transfer of nitrogen to the removal device,
optionally a device for adjusting the molar ratio of hydrogen to nitrogen to a value of about 3,
means for enriching the gas mixture with nitrogen,
one or more, consecutively connected ammonia synthesis units which preferably each comprise a compressor for compressing the gas mixture to elevated pressure, an ammonia reactor for the synthesis of ammonia from at least a part of the hydrogen and from at least a part of the nitrogen which is contained in the gas mixture, one or more heat exchangers for cooling the gas mixture and a condensing device for removal of at least a part of the ammonia, wherein in the case of several, consecutively connected ammonia synthesis units the compressors preferably present are designed each to increase the pressure of the gas mixture in comparison to the upstream ammonia synthesis unit, and wherein in the case of several, consecutively connected ammonia synthesis units the ammonia reactors are preferably positioned in a common pressure vessel or each individually in several, consecutively connected pressure vessels,
a compressor which is designed to compress the gas mixture to a pressure which is higher than the pressure in the previously traversed ammonia synthesis unit(s),
optionally a further ammonia reactor for synthesis of ammonia from at least a part of the hydrogen and from at least a part of the nitrogen which is contained in the gas mixture,
optionally one or more heat exchangers for cooling the gas mixture and a condensing device for removal of at least a part of the ammonia,
a device for combining the gas mixture with a recirculated gas mixture comprising hydrogen, nitrogen and ammonia,
an ammonia synthesis unit comprising an additional ammonia reactor for synthesis of ammonia from at least a part of the hydrogen and from at least a part of the nitrogen which is contained in the gas mixture, one or more heat exchangers for cooling the gas mixture, and one or more condensing devices for removal of at least a part of the ammonia, a compressor for compression of the gas mixture to a higher pressure, in order to compensate the pressure loss in the loop, and means for recirculating the gas mixture to the device for combining the gas mixture.

The optionally present elements of the device according to the invention are preferably, but mutually independently do not absolutely have to be, present. The elements of the device according to the invention are operatively connected, i.e. the device according to the invention comprises suitable means for the transfer of the gas mixture from one element to the next, for example suitable pipelines.

In a preferred embodiment, at least the first of the optionally several, consecutively connected ammonia synthesis units has no compressor for compression of the gas mixture to elevated pressure, and preferably the ammonia synthesis units optionally positioned thereafter still do, wherein each compressor is in each case connected upstream of the ammonia reactor. In another preferred embodiment, all ammonia synthesis units each have a compressor for compression of the gas mixture to elevated pressure, which is in each case connected upstream of the ammonia reactors.

Preferably, the circulation passed through ammonia synthesis unit and compressor has no purge, via which in conventional devices for ammonia synthesis enriched inert gases are discharged.

In a preferred embodiment, the device comprises at least one ammonia synthesis unit which comprises at least one catalyst bed which is traversed predominantly radially, preferably from outside inwards.

In a preferred embodiment, the device for preparation of a gas mixture comprises an autothermal reformer.

A further aspect of the invention relates to a device for the production of urea, comprising the device according to the invention for the production of ammonia and the additionally operatively connected elements:

a urea synthesis unit which comprises a urea synthesis reactor for the synthesis of urea from ammonia and carbon dioxide, wherein the ammonia is produced in the device for the production of ammonia, and
means for the transfer of ammonia from the device for the production of ammonia to the device for the production of urea.

Suitable urea synthesis reactors for the synthesis of urea are known to those skilled in the art. Preferably at least 40 vol. % of the ammonia produced in the device for the production of ammonia is converted to urea in the urea synthesis reactor, more preferably at least 50 vol. %, at least 60 vol. %, at least 70 vol. %, at least 80 vol. %, at least 90 vol. % or at least 99 vol. %. Preferably the whole of the ammonia produced in the device for the production of ammonia is converted to urea in the urea synthesis reactor.

In a preferred embodiment, the device for preparation of a gas mixture comprises an autothermal reformer, in which more carbon dioxide is formed than in a steam reformer.

Preferably the quantity of carbon dioxide which is formed in an autothermal reformer can be controlled by variation of reaction parameters such as for example the pressure or the steam/carbon ratio. Preferably just so much carbon dioxide is formed in the autothermal reformer that the ammonia produced in the device for the production of ammonia can preferably be completely converted to urea with carbon dioxide.

All preferred embodiments which were described above in connection with the process according to the invention also apply analogously with regard to the design and configuration of the device according to the invention and are therefore not repeated in this regard.

The device according to the invention is particularly suitable for the implementation of the process according to the invention. A further aspect of the invention therefor relates to the use of the device according to the invention for the implementation of the process according to the invention.

A further aspect of the invention relates to a process for the production of ammonium nitrate, comprising the process for the production of ammonia, wherein at least a part of the ammonia produced in the process according to the invention is used for the production of ammonium nitrate. Preferably at least 40 vol. % of the ammonia produced in the process according to the invention is used for the production of ammonium nitrate, more preferably at least 50 vol. %, at least 60 vol. %, at least 70 vol. %, at least 80 vol. %, at least 90 vol. % or at least 99 vol. %. Also preferably the whole of the ammonia produced in the process according to the invention is used for the production of ammonium nitrate.

A further aspect of the invention relates to a process for the production of nitric acid, comprising the process for the production of ammonia, wherein at least a part of the ammonia produced in the process according to the invention is used for the production of nitric acid. Preferably at least 40 vol. % of the ammonia produced in the process according to the invention is used for the production of nitric acid, more preferably at least 50 vol. %, at least 60 vol. %, at least 70 vol. %, at least 80 vol. %, at least 90 vol. % or at least 99 vol. %. Also preferably the whole of the ammonia produced in the process according to the invention is used for the production of nitric acid.

By way of example, the invention is illustrated on the basis of the diagrams. Those skilled in the art recognize that in a device according to the invention not all of the features illustrated absolutely have to be simultaneously implemented.

FIG. 1 illustrates a device according to the invention by means of which the process according to the invention can be performed. In this, a gas mixture comprising H₂, N₂, H₂O (steam), CH₄, Ar, CO, CO₂ and optionally further constituents such as for example He is firstly preferably transferred into a CO₂ scrubber (1) for removal of CO₂. The remaining gas mixture which sometimes still contains residues of CO₂ is then optionally passed into a methanation unit (2) in which CO and residues of CO₂ are converted to CH₄. These two optional measures, i.e. CO₂ scrubbing and/or methanation, preferably precede step (a) of the process according to the invention or are comprised by step (a). After this, the prepared gas mixture which sometimes inter alia still contains residues of CO, is transferred into a removal device (3), in which at least a predominant part of the CH₄, H₂O and Ar, and additionally also at least a predominant part of the CO and CO₂ are removed from the gas mixture, preferably by N₂ scrubbing. In a preferred modification, the methanation is omitted, so that the prepared gas mixture is passed directly from the CO₂ scrubber (1) into the removal device (3), which is indicated in the figures by the dotted arrow.

After this, the gas mixture cooled to low temperatures is transferred into one or more consecutively connected NH₃ synthesis units (4) which each comprise a compressor (5), an NH₃ reactor (6), one or more heat exchangers (7) and a condensing device (8) for the removal of NH₃. The NH₃ synthesis unit (4) is framed by dotted lines in FIG. 1. Index n can preferably be 1, 2, 3, 4 or 5 and thereby expresses the fact that in the case of n>1 several such NH₃ synthesis units (4) are connected sequentially one after another. In the compressor (5) the gas mixture is compressed to an elevated pressure and then transferred into the NH₃ reactor (6), in which at least a part of the H₂ contained in the gas mixture and at least a part of the N₂ contained in the gas mixture react to give NH₃. The gas mixture leaving the NH₃ reactor (6) is then cooled in the heat exchanger (7) to a comparatively low temperature, so that at least a part of the NH₃ contained in the gas mixture is condensed out in the condensing device (8) and removed from the remaining gas mixture. During this, under the conditions of the NH₃ removal optionally other substances can also be removed from the gas mixture with it. If several NH₃ synthesis units (4) are consecutively connected, then in the compressor (5) the pressure of the gas mixture is each time increased in comparison to the NH₃ synthesis unit (4) previously traversed, so that the NH₃ synthesis in the NH₃ reactor (6) of the first traversed NH₃ synthesis unit (4) takes place at lower pressure than the NH₃ synthesis in the NH₃ reactor (6) of the NH₃ synthesis unit(s) (4) traversed thereafter.

After the condensation and removal of at least a part of the NH₃ contained in the gas mixture in the condensing device (8) of the final NH₃ synthesis unit (4) traversed, the gas mixture is optionally compressed in compressor (9) to a pressure which is higher than the pressure in the previously traversed NH₃ synthesis unit(s) (4). The compressed gas mixture is then combined with a recirculated gas mixture comprising H₂, N₂ and NH₃ and passed into an NH₃ synthesis unit (10), in which in an additional NH₃ reactor at least a part of the H₂ contained in the gas mixture and at least a part of the N₂ contained in the gas mixture react to give NH₃, and the gas mixture is thereafter cooled to a comparatively low temperature, so that at least a part of the NH₃ contained in the gas mixture condenses out and is removed from the remaining gas mixture. During this, under the conditions of the NH₃ removal optionally other substances, e.g. residual quantities of inert gas, can be removed from the gas mixture with it. The remaining gas mixture is compressed in compressor (11) in order to overcome pressure losses in the loop, and as recirculated gas mixture comprising H₂, N₂ and NH₃ is combined with fresh gas mixture, before it is again transferred into an NH₃ synthesis unit (10).

Here the loop taken through NH₃ synthesis unit (10) and compressor (11) preferably has no purge (12), via which in conventional devices for NH₃ synthesis enriched inert gases are discharged. In contrast, with a device according to the invention or the process according to the invention such a purge (12) can be omitted, since the inert gases are already removed from the gas mixture beforehand, in particular in the removal device (3).

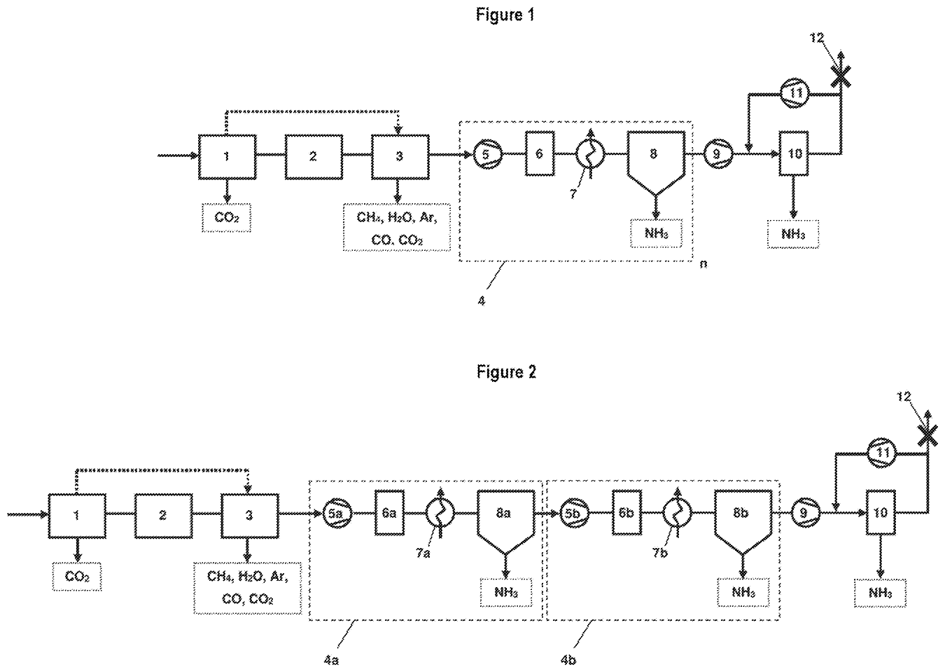

FIG. 2 diagrammatically illustrates a special case of the device shown in FIG. 1. Index n=2, so that two NH₃ synthesis units (4) are sequentially connected one after the other. The NH₃ synthesis unit (4a) is first traversed by the gas mixture which is compressed to an elevated pressure by the compressor (5a), and after this the NH₃ synthesis unit (4b) is traversed by the gas mixture which is compressed by the compressor (5b) to an elevated pressure which is higher than the pressure in the compressor (5a). In this manner, the pressure of the gas mixture is successively increased.

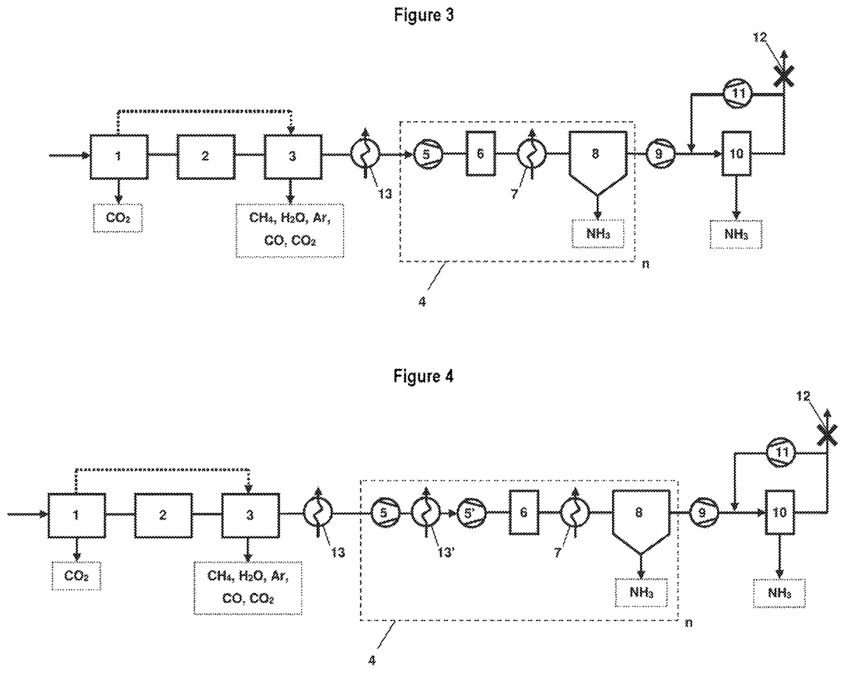

FIG. 3 diagrammatically illustrates a preferred modification of the device shown in FIG. 1 and FIG. 2 respectively, in which a heat exchanger (13) in which the gas mixture is cooled before the compression in the (first) compressor (5) is connected upstream of the respective compressor stage (5) of the (first) NH₃ synthesis unit (4).

FIG. 4 diagrammatically illustrates a preferred modification of the device shown in FIG. 3, in which a heat exchanger (13) is connected downstream of the removal device (3) and then in each of the n NH₃ synthesis unit(s) (4) a compressor (5), followed by a further heat exchanger (13′) and a further compressor (5′) is connected upstream of the respective NH₃ reactor (6). Accordingly, in this preferred embodiment, two compressor stages with an intermediate cooler are connected upstream of a reactor.

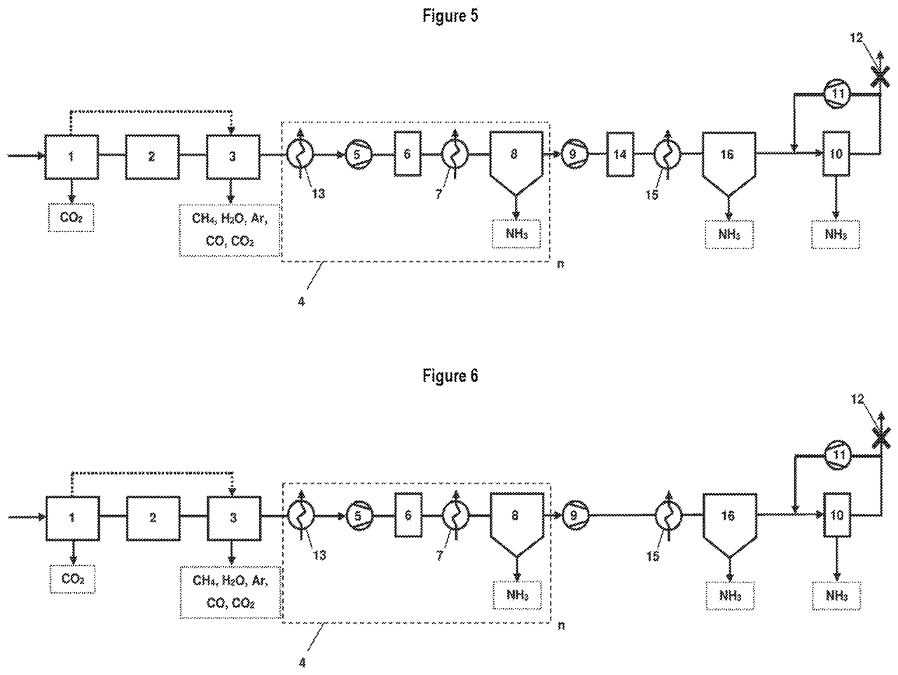

FIG. 5 diagrammatically illustrates a preferred modification of the device shown in FIG. 4, in which between compressor (9) and NH₃ synthesis unit (10) a further NH₃ reactor (14), a further heat exchanger (15) and a further condensing device (16) are positioned, which are successively traversed by the gas mixture before it is combined with the recirculated gas mixture comprising H₂, N₂ and NH₃ and fed into the NH₃ synthesis unit (10).

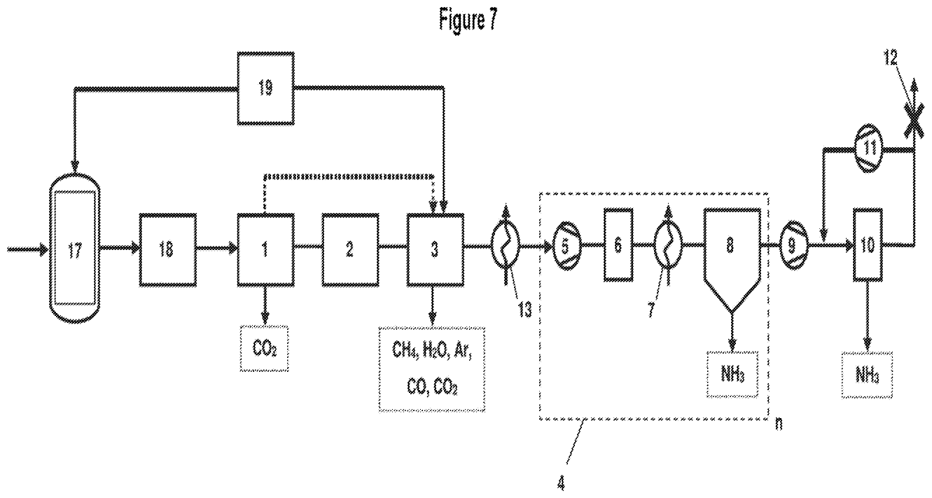

FIG. 6 diagrammatically illustrates a preferred modification of the device shown in FIG. 4, in which between compressor (9) and NH₃ synthesis unit (10), admittedly no further NH₃ reactor (see FIG. 6), but a further heat exchanger (15) and a further condensing device (16) are positioned, which are successively traversed by the gas mixture before it is combined with the recirculated gas mixture comprising H₂, N₂ and NH₃ and fed into the NH₃ synthesis unit (10).

FIG. 7 diagrammatically illustrates a modification of the device shown in FIG. 3, in which the gas mixture is prepared in a reformer (17), e.g. a two-stage steam reformer or an autothermal reformer, then is subjected to a CO conversion in a CO conversion unit (18), before it is passed into the CO₂ scrubber (1). Here the reformer (17) is operated with pure oxygen or oxygen-enriched air which is produced in an air fractionation plant (19). In this, the pure oxygen or the oxygen-enriched air can be passed into the second stage of the steam reformer or directly to the autothermal reformer, during which it is also possible that a mixing with water and/or hydrocarbons, preferably with natural gas, is firstly effected. The liquid nitrogen produced in the air fractionation plant (19) is passed to the removal device (3), in which at least a part of the CH₄, H₂O and Ar, and additionally also at least a part of the CO or CO₂ are removed from the gas mixture by N₂ scrubbing.

1.-30. (canceled)

31. A process for producing ammonia, the process comprising steps of:
(a) preparing a gas mixture comprising hydrogen, nitrogen, water, methane, carbon monoxide, and carbon dioxide;
(b) removing at least part of the water, at least part of the methane, at least part of the carbon monoxide, and at least part of the carbon dioxide from the gas mixture;
(c) synthesizing ammonia from at least part of the hydrogen and from at least part of the nitrogen contained in the gas mixture, and removing at least part of the ammonia from the gas mixture by cooling;
(d) compressing the gas mixture to a second pressure that is higher than a first pressure at which step (c) occurs;
(e) combining the gas mixture with a recirculated gas mixture comprising hydrogen, nitrogen, and ammonia; and
(f) synthesizing ammonia from at least part of the hydrogen and from at least part of the nitrogen that is contained in the gas mixture, removing at least part of the ammonia from the gas mixture by cooling, compressing the gas mixture, and returning the gas mixture as the recirculated gas mixture in step (e).

32. The process of claim 31 wherein step (c) further comprises compressing the gas mixture before the ammonia is synthesized.

33. The process of claim 31 wherein the removal of the at least part of the ammonia in at least one of step (c) or step (f) is effected by condensation.

34. The process of claim 31 wherein in step (f) the gas mixture is returned in a closed loop to step (e), from which ammonia is removed, without purge gas that contains inert gases, which are removed in a separate step.

35. The process of claim 31 wherein in step (b) the removal is effected by cooling or cooling and scrubbing by way of a nitrogen scrubber or by pressure swing adsorption.

36. The process of claim 31 wherein at least one of step (c) or step (d) further comprises cooling and then compressing the gas mixture before the compression of the gas mixture.

37. The process of claim 31 wherein in steps (c) and (f) the synthesis of ammonia occurs in ammonia synthesis units that comprise catalyst beds, wherein the gas mixture is cooled between the catalyst beds, wherein heat released between the catalyst beds by cooling is partially utilized to at least one of generate steam or preheat boiler feed water.

38. The process of claim 31 further comprising repeating step (c) before performing step (d), wherein during the repetition of step (c) the gas mixture is compressed to a third pressure that is higher than the first pressure at which step (c) occurs, wherein the second pressure to which the gas mixture is compressed in step (d) is higher than the first and third pressures.

39. The process of claim 31 further comprising converting residues of at least one of the carbon dioxide or the carbon monoxide of the gas mixture prepared in step (a) to methane by methanation before step (a), during step (a), between steps (a) and (b), or in step (b).

40. The process of claim 31 wherein step (d) after the compression comprises:
synthesizing ammonia from at least part of the hydrogen and from at least part of the nitrogen in the gas mixture; and
removing at least part of the ammonia from the gas mixture by cooling.

41. The process of claim 31 wherein the gas mixture prepared in step (a) comprises pure hydrogen and pure nitrogen.

42. The process of claim 31 wherein the gas mixture prepared in step (a) comprises a relative molar ratio of hydrogen to nitrogen of more than 3:1, wherein step (b) further comprises enriching the gas mixture with nitrogen after the removal of the at least part of the water, the at least part of the methane, the at least part of the carbon monoxide, and the at least part of the carbon dioxide.

43. The process of claim 31 wherein the gas mixture prepared in step (a) is produced by reforming of hydrocarbons, wherein the reforming occurs in a reformer that is operated with pure oxygen, with oxygen-enriched air, or with air.

44. The process of claim 43 wherein the reforming occurs in an autothermal reformer that is operated with a pressure of at least 100 bar.

45. The process of claim 43 wherein the at least part of the water, the at least part of the methane, the at least part of the carbon monoxide, and the at least part of the carbon dioxide removed in step (b) are
passed to the reformer and used as fuel gas;
mixed with the hydrocarbons that are reformed in the reformer; or
split into at least three substreams, wherein a first substream is enriched in carbon monoxide, a second substream is enriched in carbon dioxide, and a third substream is enriched in methane, and wherein
the first substream passes through a CO conversion;
the second substream is mixed with the carbon dioxide stream formed in a carbon dioxide scrubber; and
the third substream is mixed with the hydrocarbons that are reformed in the reformer.

46. The process of claim 43 wherein in step (b) the removal is effected by cooling or cooling and scrubbing, the process further comprising providing oxygen and nitrogen by way of air fractionation, wherein at least part of the oxygen provided by the air fractionation is passed into the reformer and at least part of the nitrogen provided by the air fractionation is used for the cooling in step (b).

47. The process of claim 31 wherein after step (b) the gas mixture includes at least one of:
a content of water of at most 0.05 mol. %;
a content of carbon dioxide of at most 5 ppm by volume;
a content of carbon monoxide of at most 5 ppm by volume;
a content of methane of at most 0.05 mol. %; or
a content of argon of at most 0.05 mol. %.

48. The process of claim 31 wherein
the gas mixture prepared in step (a) is produced by reforming hydrocarbons that contain no argon,
the reforming occurs in an autothermal reformer with pure oxygen or with an oxygen/nitrogen mixture that contains no argon, which is prepared by air fractionation,
the gas mixture in step (a) is subjected to a CO conversion and a carbon dioxide scrubbing, and
after the removal of the at least part of the water, the at least part of the methane, the at least part of the carbon monoxide, and the at least part of the carbon dioxide in step (b), the gas mixture is enriched with nitrogen provided by air fractionation.

49. A device for producing ammonia comprising:
a device for preparing a gas mixture comprising hydrogen, nitrogen, water, methane, carbon monoxide, and carbon dioxide;
at least one of
a first separation device for separating at least one of CO₂ or CO from the gas mixture, or
a device for converting CO and CO₂ into CH₄ and H₂O;
a second separation device for removing at least part of the water and at least part of the methane from the gas mixture;
means for enriching the gas mixture with nitrogen;
one or more sequentially-connected ammonia synthesis units, each comprising an ammonia reactor for synthesizing ammonia from at least part of the hydrogen and from at least part of the nitrogen in the gas mixture;
a first heat exchanger for cooling the gas mixture and a first condensing device for removing at least part of the ammonia;
a first compressor configured to compress the gas mixture to a pressure that is higher than a pressure in the one or more sequentially-connected ammonia synthesis units;
a device for combining the gas mixture with a recirculated gas mixture comprising hydrogen, nitrogen, and ammonia;
an ammonia synthesis unit comprising:
an ammonia reactor for synthesizing ammonia from at least part of the hydrogen and from at least part of the nitrogen in the gas mixture,
a second heat exchanger for cooling the gas mixture, and
a second condensing device for removing at least part of the ammonia;
a second compressor for compressing the gas mixture to compensate for a pressure loss due to recirculation; and
means for recirculating the gas mixture to the device for combining the gas mixture with the recirculated gas mixture.

50. The device of claim 49 wherein the first separation device comprises a CO₂ scrubber.

51. The device of claim 49 wherein the device for converting CO and CO₂ into CH₄ and H₂O comprises a methanation unit.

52. The device of claim 49 wherein the second separation device comprises a N₂ scrubber.

53. The device of claim 49 wherein at least one of the ammonia synthesis units comprises at least one catalyst bed with a predominantly radial flow.

54. The device of claim 49 wherein each of the one or more sequentially-connected ammonia synthesis units comprises a compressor connected upstream of the ammonia reactor for compressing the gas mixture, wherein if the device comprises more than one sequentially-connected ammonia synthesis unit the compressors of the sequentially-connected ammonia synthesis units are configured to increase a pressure of the gas mixture relative to each respective upstream ammonia synthesis unit.

55. The device of claim 49 wherein the device for preparing the gas mixture comprises an autothermal reformer.

56. A device for producing of urea comprising:
the device for producing ammonia as recited in claim 49;
a urea synthesis unit that comprises a urea synthesis reactor for synthesizing urea from ammonia and carbon dioxide, wherein the ammonia is prepared in the device for producing ammonia; and
means for transferring ammonia from the device for producing ammonia to the device for producing urea.
